(19) United States
(12) Patent Application Publication
(10) Pub. No.: US 2017/0281022 A1
(43) Pub. Date: Oct. 5, 2017

(54) DETECTION DEVICE, FIRST DETECTION SYSTEM, AND SECOND DETECTION SYSTEM

(71) Applicant: SEIKO EPSON CORPORATION, Tokyo (JP)

(72) Inventors: Yoshiki NAKASHIMA, Suwa-shi (JP); Yoshiki KUBOTA, Suwa-shi (JP)

(73) Assignee: SEIKO EPSON CORPORATION, Tokyo (JP)

(21) Appl. No.: 15/472,877

(22) Filed: Mar. 29, 2017

(30) Foreign Application Priority Data
Apr. 1, 2016 (JP) 2016-074031

(51) Int. Cl. A61B 5/0205; A61B 5/00; A61B 5/107

(52) U.S. Cl. CPC A61B 5/02055; A61B 5/1072; A61B 5/0008; A61B 5/6801; A61B 5/7225; A61B 2560/0214

(57) ABSTRACT

A detection device is a detection device for estimating the occurrence of an allergic reaction, and includes a detection section which noninvasively acquires information of an internal skin or a skin surface as detection data for estimating the allergic reaction. The detection section includes a skin temperature detection section which detects a skin temperature, and at least one detection section which acquires the detection data other than the skin temperature.

### BACKGROUND

### 1. Technical Field

The present invention relates to a detection device which detects an allergic reaction, and a first detection system and a second detection system each using the detection device.

### 2. Related Art

Heretofore, for allergic diseases such as atopic dermatitis, in order to specify a causative substance (allergen), a patch test or an IgE (immunoglobulin E) antibody test has been performed. Such a test is performed in each hospital or is performed by sending a sample to a specialized testing institution or the like, and it takes a long period of time until the test result is found.

JP-A-2004-267308 (Patent Document 1) discloses the following technique: a semiconductor laser is spread linearly and irradiated onto a portion to be tested, and an image is formed with irradiation spots on a light sensor, and a scanning signal is stored. Then, the stored data is analyzed for each of the portion to be tested and the other region to determine blood flow values, and a blood flow increase ratio is expressed in numerical form by displaying the ratio of both blood flow values, whereby the degree of an allergic reaction can be accurately quantitatively determined.

An allergic disease patient can obtain information on what the allergen is by a patch test, an IgE antibody test, or the like. However, for the allergic disease patient, in order to avoid the contact with the specified allergen, the information on where the patient came into contact with the specified allergen is needed. However, such information on where the patient came into contact with the allergen is not obtained by the above-mentioned test method, and therefore, the patient needs to take measures as much as possible for the activity or living environment in which the contact with the allergen is expected in a daily life. Further, it is necessary to perform the same test again for verifying the effect of the measures, however, it is difficult to perform the test every time in accordance with the daily life.

Therefore, a detection device capable of continuously estimating the occurrence of an allergic reaction, and a detection system using the detection device have been demanded.

### SUMMARY

An advantage of some aspects of the invention is to solve at least a part of the problems described above and the invention can be implemented as the following forms or application examples.

### APPLICATION EXAMPLE 1

A detection device according to this application example is a detection device for estimating the occurrence of an allergic reaction, and includes a plurality of detection sections which noninvasively acquire a plurality of pieces of information of an internal skin and/or a skin surface as detection data for estimating the allergic reaction

According to such a detection device, the device includes a plurality of detection sections which noninvasively acquire a plurality of pieces of information of an internal skin and/or a skin surface as detection data for estimating the allergic reaction. According to this, a detection device capable of acquiring detection data without giving pain to a user and also continuously estimating an allergic reaction.

### APPLICATION EXAMPLE 2

In the detection device according to the application example, it is preferred that the detection section includes a skin temperature detection section which detects a skin temperature, and at least one detection section which acquires the detection data other than the skin temperature.

According to such a detection device, in the case where there is a change the detection data other than the skin temperature, by determining whether the skin temperature when an allergic reaction occurs is within or outside a reference range, the presence or absence of the allergic reaction can be estimated.

### APPLICATION EXAMPLE 3

In the detection device according to the application example, it is preferred that the at least one detection section which acquires the detection data other than the skin temperature includes any of a blood flow rate detection section which detects a blood flow rate, a vascular diameter detection section which detects a vascular diameter, and a skin thickness detection section which detects a skin thickness.

According to such a detection device, by including any of a blood flow rate detection section, a vascular diameter detection section, and a skin thickness detection section as the at least one detection section which acquires the detection data other than the skin temperature, in the case where there is a change in the detection data of at least one of the blood flow rate, the vascular diameter, and the skin thickness, the presence or absence of an allergic reaction can be estimated by confirming the skin temperature.

### APPLICATION EXAMPLE 4

In the detection device according to the application example, it is preferred at the detection section is formed on a flat plate member.

According to such a detection device, by forming the detection section on a flat plate member, the detection device can be miniaturized or formed into a chip. According to this, for example, it becomes easy to form a detection device with portable size to be carried on the arm or the like.

### APPLICATION EXAMPLE 5

In the detection device according to the application example, it is preferred that the device includes transmission section which transmits the detection data acquired by the detection section to a predetermined device.

According to such a detection device, detection data for estimating an allergic reaction is acquired by the detection section, and the detection data is transmitted to a predetermined device by the transmission section. According to this, a detection device capable of continuously estimating an allergic reaction can be realized.

### APPLICATION EXAMPLE 6

In the detection device according to the application example, it is preferred that the acquisition of the detection data is performed at intervals within a time period in which an allergic reaction material is present.

According to such a detection device, by performing the acquisition of the detection data at intervals within a time period in which an allergic reaction material is present, for example, the occurrence of an allergic reaction in response to an immediate allergic reaction material can be reliably estimated.

### APPLICATION EXAMPLE 7

In the detection device according to the application example, it is preferred that the device includes a time measurement section, and time information measured by the time measurement section is transmitted along with the detection data.

According to such a detection device, by including a time measurement section and transmitting time information measured by the time measurement section along with the detection data, the detection data can be associated with the time information when the detection data is acquired. Further, also in the case where a predetermined device on a receiver side is not operated (when the battery is dead, or the like), the detection device side has the time information and the detection data, and therefore, the subsequent measures can be taken so that the detection data is not wasted.

### APPLICATION EXAMPLE 8

In the detection device according to the application example, it is preferred that the device includes a position specification device, and position information and time information by the position specification device are transmitted along with the detection data.

According to such a detection device, by providing the position specification device, the detection data can be associated with the position information and the time information when the detection data is acquired. Further, also in the case where a predetermined device on a receiver side is not operated (when the battery is dead, or the like), the detection device side has the detection data, the position information, and the time information, and therefore, the subsequent measures can be taken so that the detection data is not wasted. Here, the position specification device is, for example, a GPS (Global Positioning System).

### APPLICATION EXAMPLE 9

A first detection system according to this application example includes the detection device according to the application example, and a first processing device which includes a receiving section that receives the detection data transmitted from the detection device, and a processing section that processes the received detection data and stores the detection data and environmental data corresponding to the detection data in a predetermined memory region.

According to such a first detection system, by constituting the system by the detection device and the first processing device, in the first processing device, the detection data transmitted from the detection device is received by the receiving section, the received detection data is processed by the processing section, and the detection data and the environmental data corresponding to the detection data are stored in a predetermined memory region. According to this, the detection data transmitted from the detection device and the environmental data (for example, time information and position information) corresponding to the detection data can be associated with each other.

### APPLICATION EXAMPLE 10

In the first detection system according to the application example, it is preferred that the processing section estimates the presence or absence of an allergic reaction based on the received detection data of the skin temperature.

According to such a first detection system, in the case where there is a change in the detection data other than the skin temperature, by determining whether the skin temperature when an allergic reaction occurs is within or outside a reference range, the presence or absence of the allergic reaction can be estimated. For example, in the case where there is a change in the blood flow rate or the vascular diameter, and also there is a change in the skin temperature, it can be estimated that the change is caused by a reaction other than the allergic reaction (for example, an exercise or the like), and is not caused by the allergic reaction. Further, in the case where there is a change in the blood flow rate or the vascular diameter, and there is no change in the skin temperature, it can be estimated that the change is caused by the allergic reaction.

### APPLICATION EXAMPLE 11

In the first detection system according to the application example, it is preferred that the first processing device includes a position specification device, and acquires the environmental data including position information and time information by the position specification device.

According to such a first detection system, by the position specification device, the detection data and the environmental data (time information and position information) can be easily associated with each other.

### APPLICATION EXAMPLE 12

In the first detection system according to the application example, is preferred that the detection data is classified according to predetermined position information and stored.

According to such a first detection system, since the detection data is classified according to predetermined position information, for example, in the case where the detection data is classified for each region, by confirming the detection data (including the result of estimation of the presence or absence of an allergic reaction) for each region, a region where it is often estimated that an allergic reaction is present can be confirmed or the like. According to this, the first detection system can encourage a user to avoid a place where an allergic reaction is likely to occur.

### APPLICATION EXAMPLE 13

In the first detection system according to the application example, it is preferred that as the predetermined position information, the position information acquired by the position specification device or the position information received along with the detection data is used.

According to such a first detection system, as the predetermined position information, the position information acquired by the position specification device may be used or the position information received along with the detection data may be used. In particular, by using the position information received along with the detection data, even in the case where the first processing device is not operated (for example, when the device is switched off), the detection data and the position information can be acquired without any loss.

### APPLICATION EXAMPLE 14

In the first detection system according to the application example, it is preferred that the detection data is stored in a chronological order corresponding to the predetermined position information.

According to such a first detection system, by storing the detection data in a chronological order corresponding to the predetermined position information (for example, for each given region), the detection data in the region can be confirmed or compared in a chronological order.

### APPLICATION EXAMPLE 15

In the first detection system according to the application example, it is preferred that the detection data is stored when the allergic reaction is estimated to be present.

According to such a first detection system, by storing the detection data when an allergic reaction is estimated to be present, as compared with the case where all the detection data is stored, the transmission and reception time can be reduced, or the memory capacity can be reduced. Further, by associating the detection data based on which an allergic reaction is estimated to be present with the time information or the position information at that time, an allergic disease caused by the allergic reaction can be prevented.

### APPLICATION EXAMPLE 16

A second detection system according to this application example includes a plurality of first detection systems according to the application example, and a second processing device which is connected to the plurality of first processing devices constituting the plurality of first detection systems and receives and stores the detection data and the environmental data from the first processing devices.

According to such a second detection system, the system is constituted by a plurality of first detection systems and a second processing device, and the second processing device is connected to the plurality of first processing devices, and receives and stores the detection data and the environmental data from each of the first processing devices. According to this, the detection data and the environmental data obtained by the plurality of first detection systems can be stored in the second detection system.

### APPLICATION EXAMPLE 17

In the second detection system according to the application example, it is preferred that the second processing device sets a positional region where the allergic reaction estimated based on the received plurality of pieces of detection data and the environmental data, and transmits the set positional region to the first processing device of each of the first detection systems.

According to such a second detection system, by transmitting a positional region (set positional region) where the allergic reaction is estimated based on the detection data or the environmental data obtained by the plurality of first detection systems to the first detection systems (first processing devices), it becomes possible to notify a user who uses the second detection system of a place where the occurrence of an allergy is estimated including data of other users beforehand.

### APPLICATION EXAMPLE 18

In the second detection system according to the application example, it is preferred that the first processing device of the first detection system gives an alarm when acquired current position information is located in a region of the positional region received from the second processing device.

According to such a second detection system, by giving an alarm to a user who uses the second detection system, the user can be made aware of that the current position is in the positional region where an allergic reaction is estimated and is a position where an allergic symptom is likely to occur (a place where the possibility of coming into contact with an allergen is high).

### BRIEF DESCRIPTION OF THE DRAWINGS

The invention will be described with reference to the accompanying drawings, wherein like numbers reference like elements.

FIG. 1 is a schematic cross-sectional view of a detection device according to a first embodiment.

FIG. 2 is a view showing a schematic structure of a first detection system.

FIG. 3 is a view showing a schematic structure of a first detection system.

FIG. 4 is a schematic plan view of detection sections of a detection device.

FIG. 5 is a schematic cross-sectional view showing a structure of a skin temperature detection section.

FIG. 6 is a schematic cross-sectional view showing a structure of a blood flow rate detection section.

FIG. 7 is a schematic cross-sectional view showing a structure of a vascular diameter detection section.

FIG. 8 is a flowchart showing an operation of a detection device.

FIG. 9 is a flowchart showing an operation in a first processing device.

FIG. 10 is a flowchart showing an operation in a data analysis in a first processing device.

FIG, 11 is a view showing a schematic structure of a second detection system according to a second embodiment.

### DESCRIPTION OF EXEMPLARY EMBODIMENTS

Hereinafter, embodiments will be described with reference to the drawings.

### First Embodiment

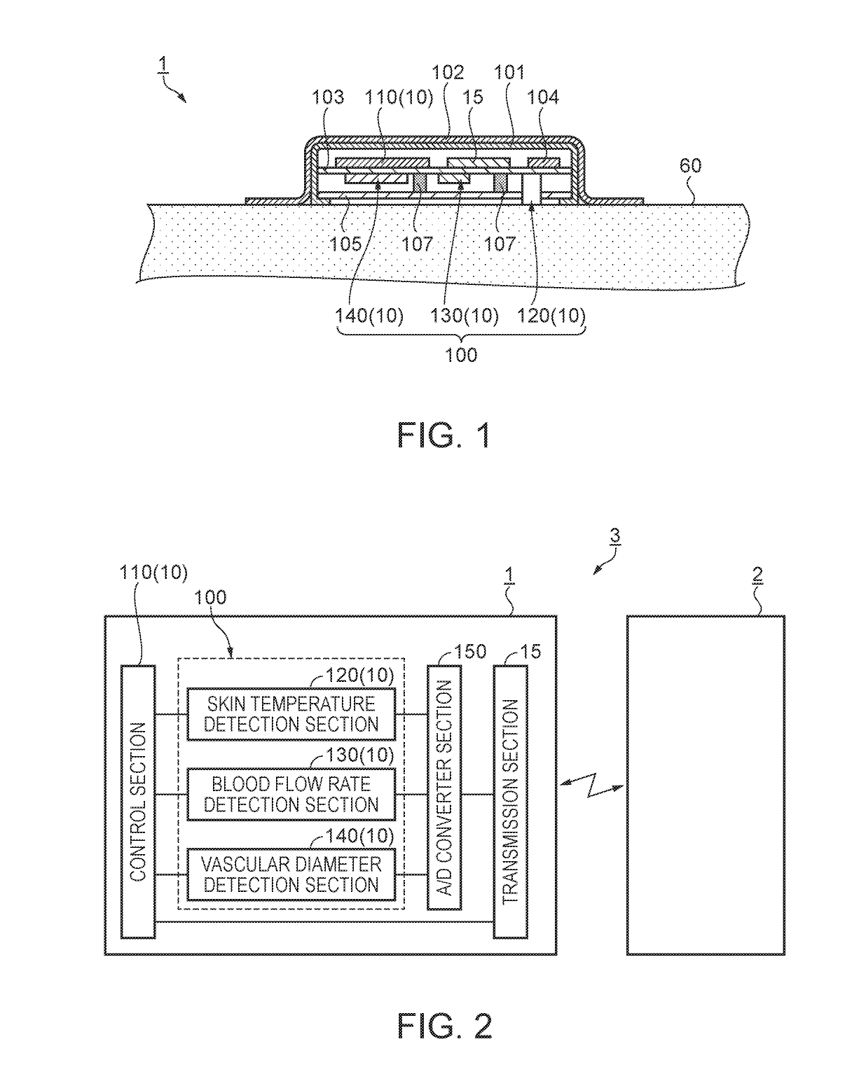

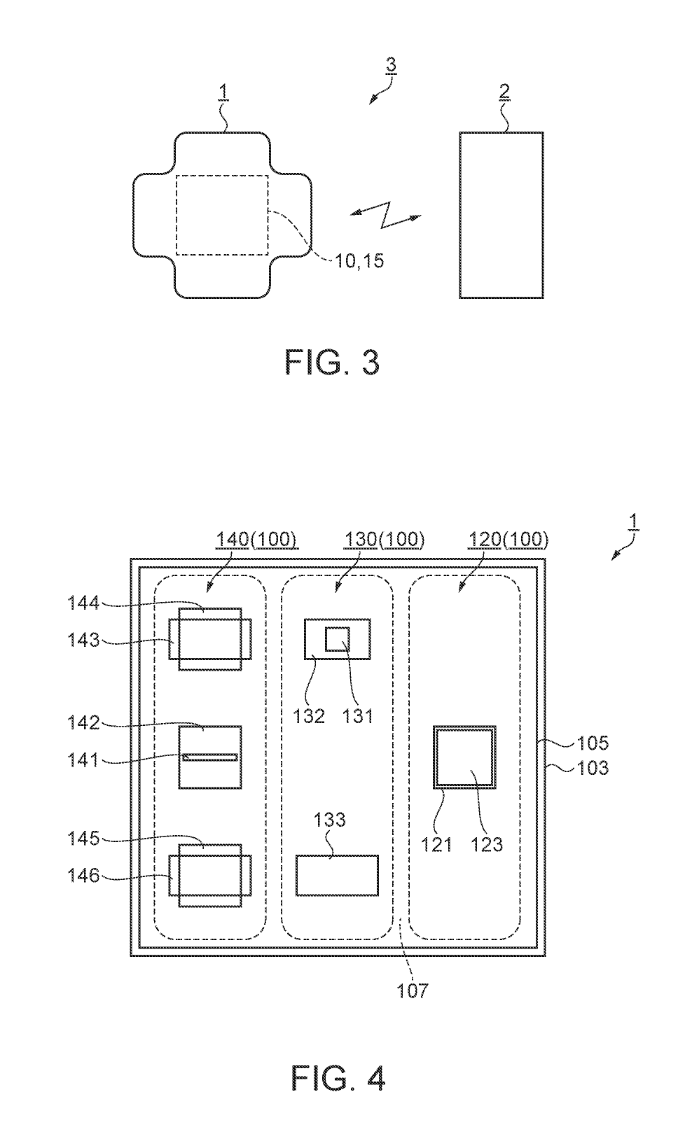

FIG. 1 is a schematic cross-sectional of a detection device 1 according to this embodiment. FIGS. 2 and 3 are views each showing a schematic structure of a first detection system 3. In FIG. 2, a schematic block diagram constituting the detection device 1 is also shown.

As shown in FIGS, 1 to 3, the detection device 1 of this embodiment is a detection device for estimating the occurrence of an allergic reaction, and acquires a plurality of pieces of information of an internal skin and a skin surface of a user as detection data for estimating the presence or absence of an allergic reaction. The detection device 1 used by being attached to a skin surface 60 (for example, arm) of a human. The detection device 1 of this embodiment is configured as a noninvasive device, and does not puncture an internal skin with a needle or the like when it is attached to the skin surface 60 of a human.

As shown in FIGS. 2 and 3, the detection device 1 is roughly constituted by a measurement section 10 and a transmission section 15. The measurement section 10 is configured to include a control section 110, three detection sections 100 (a skin temperature detection section 120, a blood flow rate detection section 130, and a vascular diameter detection section 140), an AID converter section 150, etc.

As shown in FIG. 1, the measurement section 10 and the transmission section 15 are configured to be mounted on a circuit board 103 as a flat plate member. Specifically, the detection sections 100 (the skin temperature detection section 120, the blood flow rate detection section 130, and the vascular diameter detection section 140) constituting the measurement section 10 are mounted on a lower surface side of the circuit board 103. The “lower surface side” is a side facing the skin surface 60 when the detection device 1 is fixed to the skin surface 60. Further, the control section 110, the A/D converter section 150 (not shown in FIG. 1), a power supply section 104 for driving, an operation section (not shown), etc. are mounted on an upper surface side of the circuit board 103.

The power supply section 104 is constituted by a storage battery in this embodiment. A power supply connector (not shown) for charging the storage battery is installed such that it can be exposed from the below-mentioned case 101 and adhesive pad 102. Further, operation section (not shown) is constituted by, for example, a key switch for issuing various instructions to the control section 110, and is installed such that it is exposed from the case 101 and the adhesive pad 102. The instructions of the operation section include a power-on (switch-on) instruction after the detection device W is attached to the skin, and the like.

The detection device 1 includes the case 101, the adhesive pad 102, a transparent member 105, etc. The case 101 houses the circuit board 103 (the measurement section 10, the transmission section 15, etc.) therein. The adhesive pad 102 has an adhesive surface and fixes the case 101 housing the respective constituent sections to the skin surface 60. The transparent member 105 functions as a cover of the case 101, and separates the detection device 1 from the skin surface 60 when the detection device 1 is fixed to the skin surface 60. Further, the transparent member 105 includes a function to transmit an irradiation light and a reflection light in the blood flow rate detection section 130 and the vascular diameter detection section 140. The skin temperature detection section 120 extends from the transparent member 105 and comes in contact with the skin surface 60.

Further, a partition wall 107 which separates the skin temperature detection section 120, the blood flow rate detection section 130, and the vascular diameter detection section 140 on the circuit board 103 is installed between the circuit board 103 and the transparent member 105. The detection device 1 is configured such that a light to be emitted described later or the like does not affect the respective detection sections 100 by this partition wall 107.

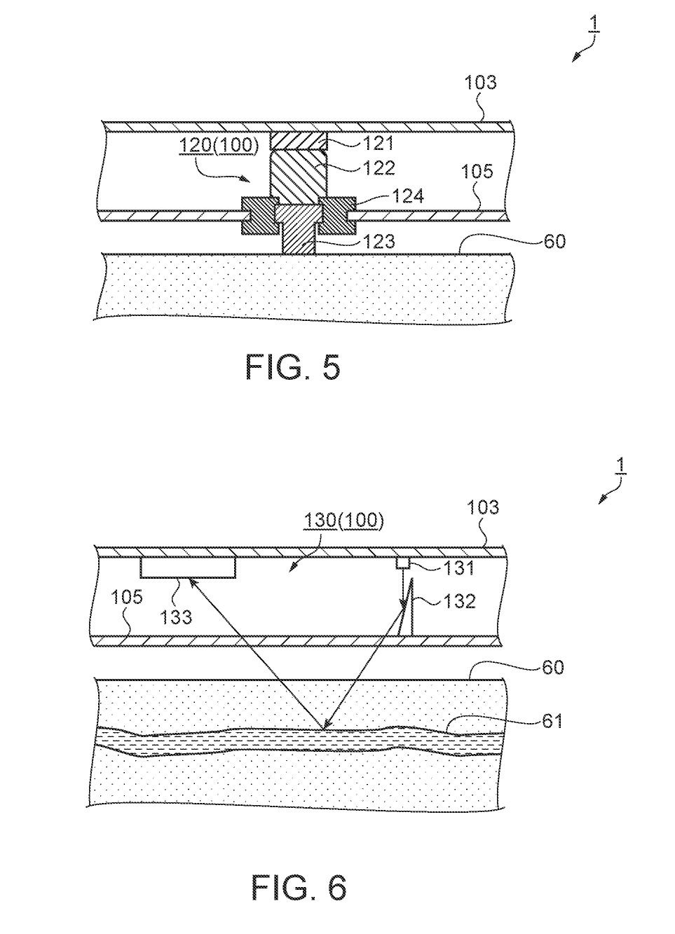

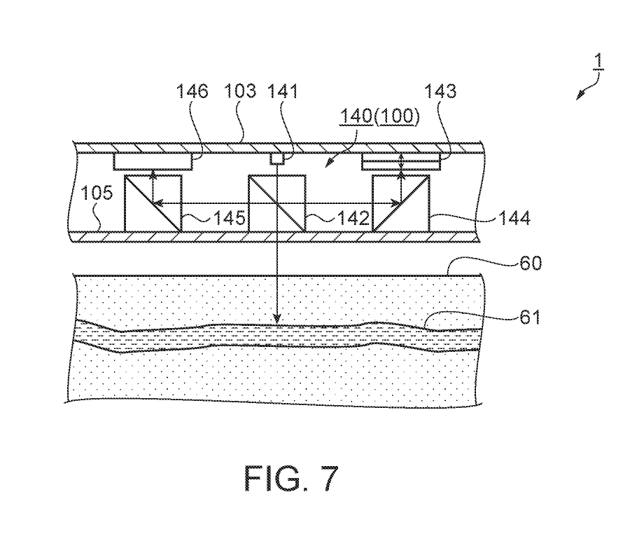

FIG. 4 is a schematic plan view of the detection sections 100 (the skin temperature detection section 120, the blood flow rate detection section 130, and the vascular diameter detection section 140) of the detection device 1. FIG. 4 is shown as a perspective view. FIG. 5 is a schematic cross-sectional view showing a structure of the skin temperature detection section 120. FIG. 6 is a schematic cross-sectional view showing a structure of the blood flow rate detection section 130. FIG. 7 is a schematic cross-sectional view showing a structure of the vascular diameter detection section 140. With reference to FIGS. 4 to 7, the structures and operations of the respective detection sections will be described.

As shown in FIG. 4, the detection sections 100 of this embodiment are mounted on the circuit board 103 having a substantially rectangular shape. Further, the detection sections 100 are configured to be interposed between the circuit board 103 and the transparent member 105 having a rectangular shape.

With reference to FIGS. 4 and 5, the structure and operation of the skin temperature detection section 120 will be described.

The skin temperature detection section 120 is constituted by a thermistor element 121, a paste section 122, and a heat-transfer member 123. The thermistor element 121 is mounted on the circuit board 103, and the heat-transfer member 123 extends from the transparent member 105, and the paste section 122 is configured to be interposed between the thermistor element 121 and the heat-transfer member 123.

The heat-transfer member 123 is constituted by a member made of a metal and having a higher thermal conductivity, and is configured such that when the detection device 1 fixed to the skin surface 60, a tip portion of the heat-transfer member 123 comes into contact with the skin surface 60 so as to transfer the temperature of the skin surface. The heat-transfer member 123 is fixed to the transparent member 105 by an insulating member 124 made of a resin, and is configured such that the heat transferred to the heat-transfer member 123 does not escape to the transparent member 105. The paste section 122 is constituted by a heat-transfer paste having a high thermal conductivity, and transfers heat transferred from the heat-transfer member 123 to the thermistor element 121. Incidentally, for example, a resistance value corresponding to the heat transferred to the thermistor element 121 is stored in a memory section (not shown). In this embodiment, a skin temperature or the like is calculated by the processing (the execution of a program) of the processing section of the first processing device 2 described later.

With reference to FIGS. 4 and 6, the structure and operation of the blood flow rate detection section 130 will be described.

The blood flow rate detection section 130 is formed next to the skin temperature detection section 120. The blood flow rate detection section 130 is configured by adopting a Doppler method using a far-red light. The blood flow rate detection section 130 is constituted by a light source 131, a reflecting mirror 132, and a light-receiving element 133. The light source 131 and the light-receiving element 133 are mounted on the circuit board 103, and the reflecting mirror 132 is fixed to the transparent member 105 facing the light source 131 so as to receive a light from the light source 131.

The light source 131 is constituted by a semiconductor laser which emits a far-red light. The light emitted from the light source 131 is reflected by the reflecting mirror 132, and transmitted through the transparent member 105, and then, irradiated on the skin surface 60. The light irradiated on a biological tissue is scattered by a resting tissue of a living body. The frequency of the scattered light is the same as the frequency of the irradiation light. Further, the light scattered by blood cells such as ed blood cells moving in a blood vessel 61 by the irradiation light is slightly Doppler-shifted with respect to the moving speed. A light including a beat signal obtained by superimposing the frequency of the light scattered from the resting issue on the frequency of the shifted light is received by the light-receiving element 133 through the transparent member 105. The received light here is stored in the memory section (not shown). In this embodiment, a blood flow rate or the like is calculated by analyzing the frequency of the received light by the processing the execution of a program) of the processing section of the first processing device 2 described later.

With reference to FIGS. 4 and 7 the structure and operation of the vascular diameter detection section 140 will be described.

The vascular diameter detection section 140 is formed next to the blood flow rate detection section 130. The vascular diameter detection section 140 is configured by adopting optical coherence tomography using a far-red light. The vascular diameter detection section 140 is constituted by a light source 141, a half mirror 142, a movable mirror 143, reflecting mirrors 144 and 145, and a light-receiving element 146.

The light source 141 and the light-receiving element 146 are mounted on the circuit board 103, and the half mirror 142, and the reflecting mirrors 144 and 145 are fixed to the transparent member 105. Further, the movable mirror 143 is installed on the circuit board 103 facing the reflecting mirror 144 such that the distance between the movable mirror 143 and the reflecting mirror 144 can be changed. Specifically, the members are installed such that the light source 141 faces the half mirror 142, the movable mirror 143 faces the reflecting mirror 144, and the light-receiving element 146 faces the reflecting mirror 145.

The light source 141 is configured as a line light source adopting an LED (Light Emitting Diode) which emits a far-red light. The light source 141 may be configured as a plane light source other than the line light source.

The half mirror 142 is installed at an angle of 45° on the transparent member 105. A light emitted from the light source 141 is branched by the half mirror 142 into an optical path transmitted and irradiated onto the skin surface 60 and an optical path reflected on the movable mirror 143 side. The light transmitted through the half mirror 142 is transmitted through the transparent member 105 and reflects an image corresponding to the image of a blood vessel in a living body. This light returns to the half mirror 142 again, and is reflected by the half mirror 142, and thereafter reflected by the reflecting mirror 145, and then is incident on the light-receiving element 146.

The light initially reflected by the half mirror 142 is reflected by the reflect a mirror 144, and is incident on and reflected, by the movable mirror 143. This light is reflected by the reflecting mirror 144, transmitted through the half mirror 142, and thereafter reflected by the reflecting mirror 145, and then is incident on the light-receiving element 146. The two lights incident on the light-receiving element 146 interfere with each other due to a difference in the optical path length, whereby an interference image obtained. Further, by changing the optical path length by moving the movable mirror 143, the interference, image is changed. The received interference image and the changed interference image are stored in the memory section (not shown). In this embodiment, a vascular diameter or the like is calculated by the processing (the execution of a program) of the processing section of the first processing device 2 described later.

Incidentally, a configuration in which each detection section 100 includes a control section which controls the above-mentioned respective operations or a memory section may be adopted.

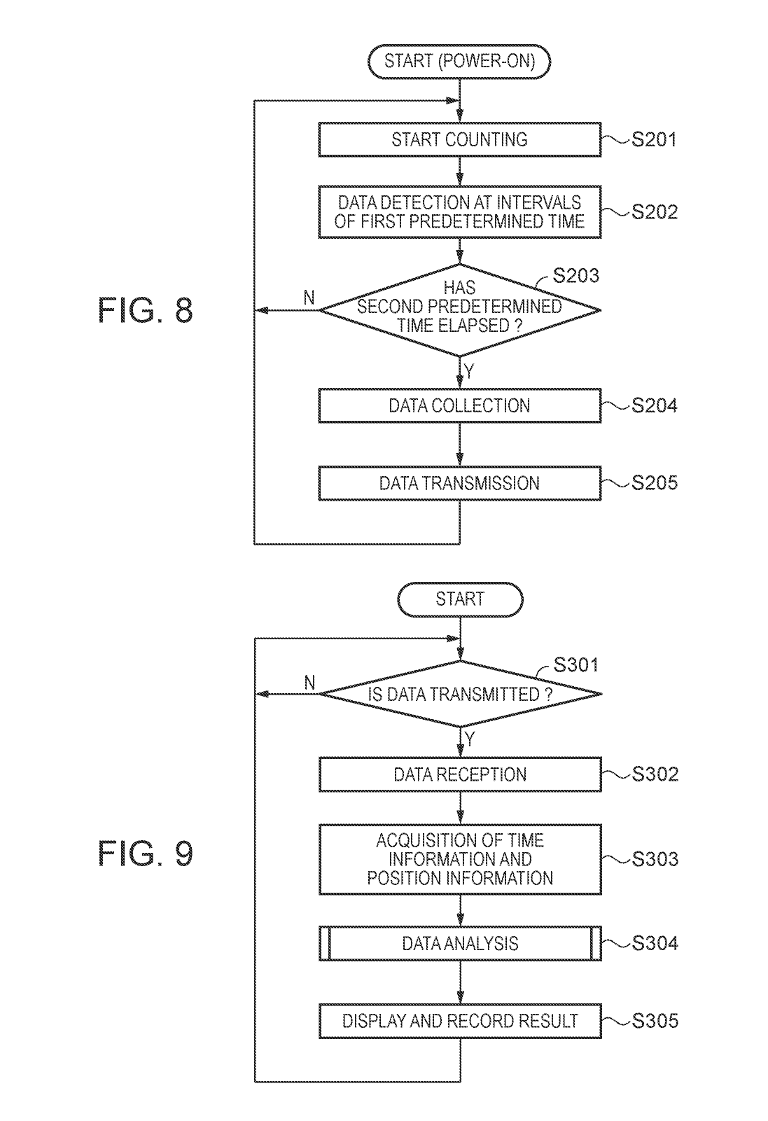

FIG. 8 is a flowchart showing an operation of the detection device 1.

After the power supply of the detection device 1 is turned on, the control section 110 reads a program for processing stored in the memory section (not shown) and starts an operation. Then, the control section 110 of a timer (not shown) included in the detection device 1, and starts counting first and second predetermined times (Step S201). Thereafter, the process shifts to Step S202, and data detection is performed at intervals of the first predetermined time. Specifically, the control section 110 operates the respective detection sections 100, and each piece of data i s detected at intervals of 30 seconds as the first predetermined time and stored in the memory section. Then, the control section 110 detects and stores data six times in total at intervals of 30 seconds in this embodiment. The first predetermined time is not limited to 30 seconds and can be appropriately set.

Subsequently, in Step S203, the control section 110 determines whether or not the second predetermined time has elapsed. The second predetermined time is 3 minutes in this embodiment. In the case where 3 minutes have elapsed (Yes in Step S203), the process shifts to data collection in Step S204. In the case where 3 minutes have not elapsed (No in Step S203), the process shifts to Step S201, and then, data detection is continued at intervals of the first predetermined time.

In the case where the process shifts to Step S204, the control section 110 collects the detection data of the respective detection sections 100 detected and stored six times in total at intervals of 30 seconds. Thereafter, the process shifts to Step S205, and the control section 110 places the collected respective pieces of data in the order by attaching an identification tag thereto, and transmits the data to the first processing device 2 through the transmission section 15. After the transmission, the control section 110 returns the process to start counting (Step S201), and repeats a series of operations: driving the respective detection sections 100 at intervals of 30 seconds, storing detection data six times in total, and transmitting the respective pieces of detection data to the first processing device 2 at intervals of 3 minutes.

In this embodiment, the detection device 1 detects a change in the internal skin or the skin surface 60 by the effect of histamine or the like which is an allergic reaction material. Histamine is a material which is produced in the end in an immediate allergic reaction and directly causes disease symptoms such as itch or redness by dilating a blood vessel, or the like. It is known that histamine circulates in the body (in the blood) and disappears (is decomposed) in about 5 minutes or so after generation. Therefore, in this embodiment, a configuration in which a change caused by an immediate allergic reaction is captured by performing detection at intervals of 3 minutes as an interval within a time period in which an allergic reaction material is present is adopted. However, the interval is not limited to 3 minutes as long as the interval is set within a time period in which an allergic reaction material is present. Further, in consideration of leukotrienes, interleukin-4, interleukin-13, E (immunoglobulin E) eosinophils, etc. other than histamine, the interval may also be appropriately set within a time period in which such an allergic reaction material is present.

The first detection system 3 of this embodiment is constituted by the detection device 1 and the first processing device 2 as shown in FIGS. 2 and 3. The detection device transmits the acquired detection data to the first processing device 2 as the predetermined device through wireless communication. As the transmission section 15, an appropriate wireless communication module which can perform short-range wireless communication utilizing an NFC (Near Field Communication), a Bluetooth (registered trademark) standard, a Wi-Fi (registered trademark) standard, or the like, wireless data communication utilizing a mobile phone line such as a CDMA2000 system, a W-CDMA system, or an LTE (Long term Evolution) system, or the like can be used as long as wireless communication between the transmission section 15 and the first processing device 2 can be performed.

The first processing device 2 is constituted by a portable-type device, a so-called smartphone, tablet, PC (personal computer), or the like, and is carried along with the detection device 1 by a user who uses the detection device 1. The first processing device 2 includes a receiving section (not shown) and a processing section (not shown). The receiving section receives the detection data transmitted from the detection device 1, and the processing section processes the received detection data, and stores the detection data and environmental data (for example, the below-mentioned time information or position information) corresponding to the detection data in a predetermined memory region. The first detection system 3 performs processing with respect to the detection data transmitted from the detection device 1 according to a computer program executed by the processing section (CPU: Central Processing Unit) included in the first processing device 2.

FIG. 9 is a flowchart showing an operation in the first processing device 2.

As shown in. FIG. 9, in the case where the program is started to be executed, in Step S301, the first processing device 2 determines whether or not the data is transmitted from the detection device 1. Specifically, whether or not the detection data is transmitted is determined by whether or not the identification tag attached to the transmitted detection data is recognized. Then, in the case where the identification tag is recognized (Yes in Step S301), the transmitted detection data is received (data reception in Step S302). In the case where the identification tag is not recognized (No in Step S301) the device waits for the detection data to be transmitted.

In the case where the detection data is received in Step S302, subsequently, position information time information are acquired (Step S303). Here, the first processing device 2 includes a GPS (Global Positioning System) (not shown) as the position specification device. Then, by operating this GPS, current time information is acquired along with current position information.

Subsequently, the received detection data is analyzed (data analysis in Step S304). The data analysis in Step S304 will be described later.

In the case where the data analysis is completed, the process shifts to Step S300, and the analysis result is displayed and recorded. The result of estimation of the presence or absence of an allergic reaction or the acquired time information is classified based on the predetermined position information, and further classified in a chronological order, and then displayed and stored. Specifically, in this embodiment, a region (positional region) is set by a predetermined radius (for example, 10 m) using the position where the position information is acquired as the center. Then, with respect to the set region, the determined estimation result or the acquired time information is associated and classified, and displayed on a display section (not shown) or stored in the memory section (not shown).

After the region is set, in the case where the position information to be acquired next time is located within the range of the set region, it is processed by being associated with the estimation result and time information corresponding to this region and classified. Further, as the display of the result of estimation of the presence or absence of an allergic rear; ion, in this embodiment, for example, “A” is displayed in the case where an allergic reaction is “present” and “B” is displayed in the case where an allergic reaction is “absent”, and so on.

After the analysis result is displayed and recorded, the process shifts to Step S301, and the device waits for the detection data to be transmitted.

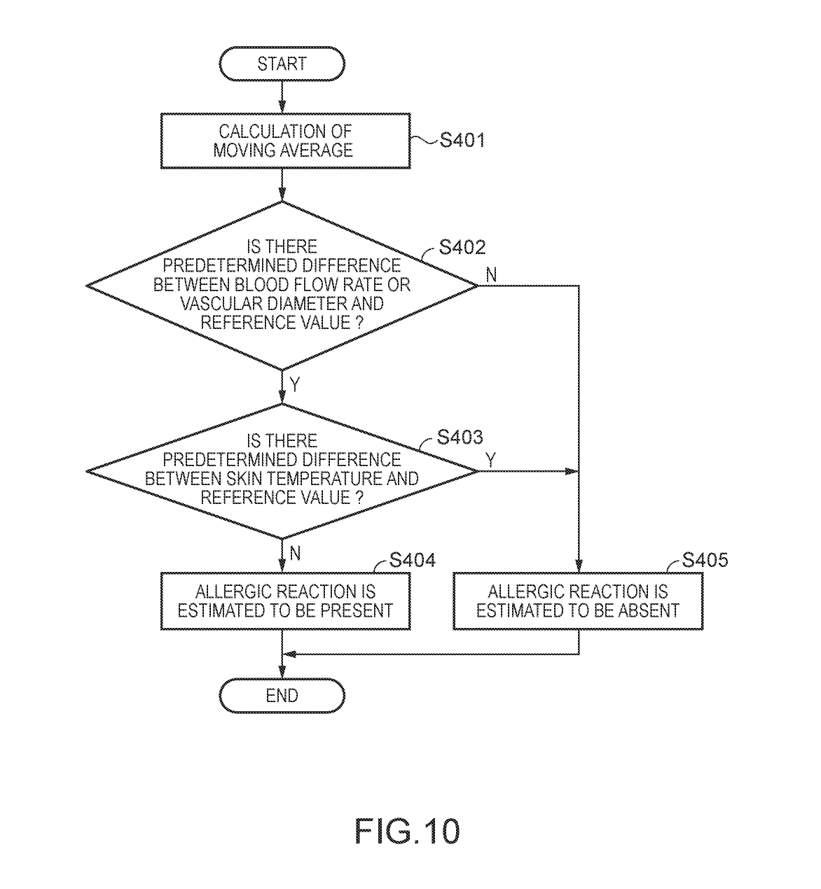

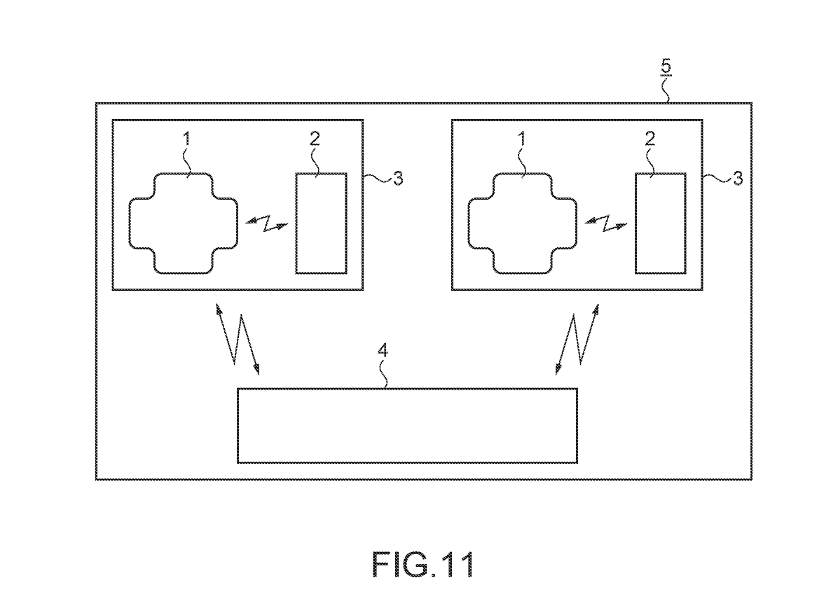

FIG. 10 is a flowchart showing an operation in a data analysis in the first processing device 2.

As shown in FIG. 10, in the case where a program for a data analysis is started to be executed, the first processing device 2 calculates a moving average of the received detection data of each detection section 100 (Step S401). Specifically, in this embodiment, an average value is calculated for received 6 pieces of detection data in the skin temperature detection section 120. Further, an average value is calculated for received 6 pieces of detection data in the blood flow rate detection section 130. Further, an average value is calculated for received 6 pieces of detection data in the vascular diameter detection section 140.

Subsequently, it is determined whether or not there is a predetermined difference between the value of the blood flow rate or the vascular diameter and each reference value by comparison (Step S402). Specifically, it is determined whether or not there is a predetermined difference between the reference value in each of the blood flow rate detection section 130 and the vascular diameter detection section 140 with the average value of each of the blood flow rate and the vascular diameter (the average value showing the blood flow rate and the average value showing the vascular diameter) calculated in Step S401 by comparison. The predetermined difference may be input and set as the initial setting, or may be set according to a program as value determined initially. The predetermined difference shows the allowable range of variation of each value.

Further, the reference value of each of the blood flow rate and the vascular diameter may be set initially by executing a program for setting the reference value included in the first processing device 2 so as to detect the user's own normal blood flow rate and normal vascular diameter by the first detection system 3. Further, the set reference value is stored and may be used as a table for comparison.

In Step S402, in the case where it is determined that there is a predetermined difference between at least one of the value of the blood flow rate and the value of the vascular diameter and each reference value by comparison (Yes in Step S402), subsequently, the process shifts to Step S403. Further, in the case where it is determined that there is no predetermined difference between the value of the blood flow rate or the vascular diameter and the reference value by comparison (No in Step S402), subsequently, the process shifts to Step S405.

In Step S403, it is determined whether or not there is a predetermined difference between the value of the skin temperature and a reference value by comparison. Specifically, it is determined whether or not there is a predetermined difference between a reference value in the skin temperature detection section 120 and an average value of the skin temperature (an average value showing the skin temperature) calculated in Step 5401 by comparison. The predetermined difference may be input as the initial setting, or may be set according to a program as a value determined initially in the same manner as described above.

Further, the reference value of the skin temperature may be set initially by executing a program for setting the reference value included in the first processing device 2 so as to detect the user's own normal skin temperature by the first detection system 3 in the same manner as described above. Further, the set reference value is stored and may be used as a table for comparison.

In Step 8403, in the case where it is determined that there is no predetermined difference between the value of the skin temperature and the reference value by comparison (No in Step S403), subsequently, the process shifts to Step S404. Further, in the case where it is determined that there is a predetermined difference between the value of the skin temperature and the reference value by comparison (Yes in Step S403), subsequently, the process chits to Step S405.

In the case where the process shifts to Step S404, when there is a predetermined difference between the value of the blood flow rate or the vascular diameter and each reference value by comparison, and also there is no predetermined difference between the value of the skin temperature and the reference value by comparison, the detection data detected this time estimates that an allergic reaction is present.

As described above, in the case where there is no predetermined difference between the value of the blood flow rate or the vascular diameter and each reference value by comparison (No in Step S402), in Step S405, the detection data detected this time estimates that an allergic reaction is absent. Further, also in the case where there is a predetermined difference between at least one of the value of the blood flow rate and the value of the vascular diameter and each reference value by comparison (Yes in Step S402), when it is determined that there a predetermined difference between the value of the skin temperature and the reference value by comparison (Yes in Step S403), in Step S405, the detection data detected this time estimates that an allergic reaction is absent.

In the case where the presence or absence of an allergic reaction is determined in Steps S404 and S405, the process shifts to Step S305 in the flowchart shown in FIG. 9. The operation in Step S305 is as described above.

The premise of the estimation of the presence or absence of an allergic reaction is considered as follows.

In the case where a user performed a normal exercise, the blood flow rate naturally increases and the vascular diameter also increases regardless of an allergic reaction. Further, the body temperature (skin temperature) also naturally increases. On the other hand, in the case where an allergic reaction is present (an allergic reaction has occurred), the blood flow rate increases and also the vascular diameter increases along with this, however, the body temperature (skin temperature) is constant (within the allowable range, that is, within the predetermined difference).

Therefore, in the data analysis of the detection data, in the case where the blood flow rate increases and the vascular diameter also increases, when the skin temperature does not change, an allergic reaction is estimated to be present. Further, in the case where the blood flow rate increases and the vascular diameter also increases, when the skin temperature also increases, an allergic reaction is estimated to be absent on the presumption that the chancre is caused by an exercise. In other words, the presence or absence of an allergic reaction is estimated based on the detection data of the skin temperature.

Further, the average values (the average value of the skin temperature, the average value of the blood flow rate, and the average value of the vascular diameter) of each detection section 100 calculated in the calculation of the moving average (Step S401) are displayed on the display section and also stored in the memory section in Step S305 in the flowchart shown in FIG. 9.

According to the above-mentioned embodiment, the following effects are obtained.

(1) According to the detection device 1 of this embodiment, a plurality of detection sections 100 (the skin temperature detection section 120, the blood flow rate detection section 130, and the vascular diameter detection section 140) which noninvasively acquire a plurality of pieces of information of the internal skin and/or the skin surface as detection data for estimating an allergic reaction are included. According to this, the detection device 1 capable of acquiring detection data without giving pain to a user can be realized and also continuously estimating an allergic reaction.

(2) According to the detection device of this embodiment, by using the skin temperature which is considered not to abruptly change when an allergic reaction occurs as the base, in the case where there is a change in each piece of the detection data of the blood flow rate and the vascular diameter other than the skin temperature, the presence or absence of an allergic reaction can be estimated by confirming the skin temperature in this embodiment.

(3) According to the detection device 1 of this embodiment, by including the blood flow rate detection section 130 and the vascular diameter detection section 140 as at least one detection section 100 which acquires detection data other than the skin temperature, in the case where there is a change in the detection data of at least one of the blood flow rate and the vascular diameter, the presence or absence of an allergic reaction can be estimated by confirming the skin temperature. In this embodiment, the blood flow rate and the vascular diameter are detected, the accuracy in the case where the presence or absence of an allergic reaction is estimated can be improved.

(4) According to the detection device 1 of this embodiment, by forming the three detection sections 100 on the circuit board 103 as the flat plate member, the detection device 1 can be miniaturized or formed into a chip. According to this, for example, it becomes easy to form the detection device 1 with a portable size to be carried on the arm or the like.

(5) According to the detection device 1 of this embodiment, detection data for estimating an allergic reaction is acquired by the detection sections 100, and the detection data is transmitted to the first processing device 2 by the transmission section 15. According to this, the detection device 1 capable of continuously estimating an allergic reaction can be realized.

(6) According to the detection device 1 of this embodiment, the acquisition of the detection data is performed at intervals within a time period in which an allergic reaction material is present in this embodiment, in consideration of histamine which is an immediate allergic reaction material, the detection data is acquired at intervals of 3 minutes. According to this, the occurrence of the allergic reaction can be reliably estimated.

(7) According to the first detection system 3 of this embodiment, by constituting the system by the detection device 1 and the first processing device 2, in the first processing device 2, the detection data transmitted from the detection device 1 is received by the receiving section, the received detection data is processed by the processing section, and the detection data and the environmental data corresponding to the detection data are stored in a predetermined memory region (memory section). According to this, the detection data transmitted from the detection device 1 and the environmental data (for example, time information and position information) corresponding to the detection data can be associated with each other.

(8) According to the first detection system 3 of this embodiment, by using the skin temperature which is considered not to abruptly change when an allergic reaction occurs as the base, in the case where there is a change in the detection data (the detection data of the blood flow rate or the detection data of the vascular diameter) other than the skin temperature, the presence or absence of an allergic reaction can be estimated by confirming the skin temperature. In this embodiment, in the case where there is a change in the blood flow rate or the vascular diameter, and when there is also a change in the skin temperature, it is estimated that the change is caused by a reaction other than the allergic reaction (for example, an exercise), and is not caused by the allergic reaction. In addition, in the case where there is a change in the blood flow rate or the vascular diameter, and when there is no change in the skin temperature, it is estimated that the change is caused by the allergic reaction.

(9) According to the first detection system 3 of this embodiment, a GPS is included, and therefore, the detection data and the environmental data (time information and position information) can be easily associated with each other.

(10) According to the first detection system 3 of this embodiment, the detection data is classified according to predetermined position information, and therefore, in the case where the detection data is classified for each region, by confirming the detection data (including the result of estimation of the presence or absence of an allergic reaction) for each region, a region where it is often estimated that an allergic reaction is present can be confirmed or the like. According to this, the first detection system 3 can encourage a user to avoid a place where an allergic reaction is likely to occur.

(11) According to the first detection system 3 of this embodiment, by storing the detection data in a chronological order corresponding to the predetermined position information (for each region), the detection data (in this embodiment, the data (average values) of the skin temperature, the blood flow rate, and the vascular diameter, and the result of estimation of the presence or absence of an allergic reaction) in the region can be confirmed or compared in a chronological order.

### Second Embodiment

FIG. 11 is a view showing a schematic structure of a second detection system 5 according to a second embodiment.

The second detection system 5 of this embodiment is a system configured to include a plurality of first detection systems 3 of the first embodiment described above and a second processing device 4 which is connected to the plurality of first detection systems 3.

The second processing device 4 is constituted by a so-called server or the like. The second processing device 4 is connected to a plurality of first processing devices 2 constituting the plurality of first detection systems 3, and each piece of detection data (in this embodiment, the data (average values) of the skin temperature, the blood flow rate, and the vascular diameter, and the result of estimation of the presence or absence of an allergic reaction) and environmental data (position information and time information) from the first processing devices 2 is received and stored. In this embodiment, in the flowchart shown in FIG. 9 in the first embodiment, after the data analysis (Step S304), data is transmitted to the second processing device 4 from the first processing device 2.

The second processing device 4 and the first processing device 2 are connected in both directions through a telecommunication line such as a wireless LAN line, a mobile phone line, or an Internet line. In the second detection system 5, a plurality of pieces of data of a plurality of users who use the first detection system 3 are integrated, stored, and managed. According to this, the plurality of pieces of data of the plurality of users can be utilized. In the second detection system 5, the second processing device 4 and the plurality of first processing devices 2 perform an operation by a web application.

The second processing device 4 set a positional region (region) where an allergic reaction is estimated to be present based on the plurality of pieces of the respective detection data and environmental data (positional information and time information) received from the plurality of first processing devices 2 and also classifies the region in a chronological order. Then, the region where an allergic reaction is estimated to be present and the time information are transmitted to each of the first processing devices 2. The user who uses the first processing device 2 can confirm the received data.

Further, in the second detection system 5, the first processing device 2 acquires map information from the second processing device 4, and the region where an allergic reaction is estimated to be present is mapped on the acquired map, and can be displayed through the display section. The mapping of the region where an allergic reaction is estimated to be present on the map may be performed by the second processing device 4. The user can confirm a place where an allergic reaction is mated to be present (a place where the possibility of coming into contact with an allergen is high) by confirming the map displayed on the display section of the first processing device 2,

Further, in the case where the first processing device 2 includes an alarm section constituted by an element which outputs a sound, an element which emits a light, an element which generates a vibration, or the like, when the current position of the user is located in the region where an allergic reaction is estimated to be present, it is possible to give an alarm that the possibility of coming o contact with an allergen is high to the user through the alarm section. Further, also in the case where the current position of the user is located near the region where an allergic reaction is estimated to be present, it is possible to give an alarm to the user through the alarm section beforehand.

According to the above-mentioned embodiment, the same effects as those of the first embodiment can be exhibited, and also the following effects are obtained.

(1) According to the second detection system 5 of this embodiment, the system is constituted by the plurality of first detection systems 3 and the second processing device 4, and the second processing device 4 is connected to the plurality of first processing devices 2, detection data (data (average values) of the skin temperature, the blood flow rate, and the vascular diameter, and the result of estimation of the presence or absence of an allergic reaction) and environmental data from each of the first processing devices 2 are received and stored. According to this, the detection data and the environmental data obtained by the plurality of first detection systems 3 can be stored in the second processing device 4.

(2) According to the second detection system 5 of this embodiment, by transmitting a region where an allergic reaction is estimated to be present (a set region) to the first processing device 2 based on the detection data and the environmental data obtained by the plurality of first detection systems 3, it becomes possible to notify the user who uses the second detection system 5 of a place where the occurrence of an allergy is estimated including data of the other users beforehand. According to this, the user can utilize the data of the other users, and can expand the range of activity in a daily life with peace of mind.

(3) According to the second detection system 5 of this embodiment, by giving an alarm to the user who uses the second detection system 5, it is possible to make the user aware of that the current position is in a region where an allergic reaction is estimated to be present and is a position where an allergic symptom is likely to occur (a place where the possibility of coming into contact with an allergen is high) According to this, it is possible to eliminate the inconvenience of the user that the user acts while always confirming the first processing device 2 in a daily life.

The invention is not limited to the above-mentioned embodiments and may be taken into practice by adding various changes and modifications thereto without departing from the gist of the invention. Hereinafter, modification examples will be described.

In the above-mentioned respective embodiments, the detection device 1 may include a time measurement section. In this case, time information at the initial time point of acquisition of the detection data is acquired, and the detection data and the time information may be transmitted to the first processing device 2 from the transmission section 15. In this case, the first processing device 2 receives the detection data and the time information, and thereafter, processing can be performed by the first processing device 2. The detection device 1 may include a memory section, and the acquired detection data and the time information may be stored there. According to this configuration, also in the case where the first processing device 2 is not operated (when the battery is dead, the device fails to be turned on, or the like), the detection data and the time information at the time point of detection can be stored in the detection device 1, and therefore, the subsequent measures can be taken so that the detection data is not wasted. According to this, the convenience of the detection device 1 is improved.

In the above-mentioned respective embodiments, the detection device 1 may include a GPS. In this case, positional information and time information at the initial time point of acquisition of the detection data by the detection device 1 are acquired. Then, the detection data, the positional information, and the time information may be transmitted to the first processing device 2 from the transmission section 15. In such a case, the first processing device 2 receives the detection data, the positional information, and the time information, and thereafter, processing can be performed by the first processing device 2. The detection device 1 may include a memory section, and the detected detection data, the positional information, and the time information may be stored there. According to this configuration, also in the case where the first processing device 2 is not operated, the detection data, and the positional information and the time information at the time point of detection can be stored in the detection device 1, and therefore, the subsequent measures can be taken so that the detection data is not wasted. According to this, the convenience of the detection device 1 is improved.

In the above-mentioned first embodiment, in the data analysis (Step S304), the presence or absence of an allergic reaction is estimated, and the detection data is stored in the memory section. However, the storage (preservation) of detection data may storage of only detection data when an allergic reaction is estimated to be present. According to this, as compared with a case where all the detection data is stored, the memory capacity can be reduced, and also the transmission and reception time can be reduced. Further, by associating the detection data based on which an allergic reaction is estimated to be present with the time information or the position information at that time, an allergic disease caused by the allergic reaction can be prevented.

In the above-mentioned respective embodiments, the skin temperature detection section 120, the blood flow rate detection section 130, and the vascular diameter detection section 140 constituting the detection sections 100 are included, and all the detection sections 100 are made to function. However, the configuration is not limited thereto, and a configuration in which the skin temperature detection section 120 and at least one of the blood flow rate detection section 130 and the vascular diameter detection section 140 are made to function may be adopted. Further, other than the skin temperature detection section 120, the blood flow rate detection section 130, and the vascular diameter detection section 140 in the above-mentioned respective embodiments, another detection section such as a skin thickness (a distance from the skin surface 60 to the internal blood vessel 61) detection section is further provided and may be made to function. According to such a detection device, the accuracy of estimation of the detection of an allergic reaction can be improved.

In the first detection system 3 of each of the above-mentioned embodiments, the first processing device 2 calculates a moving average for each piece of the detection data received from the detection device 1 (Step S401 in FIG. 10). However, the configuration is not limited thereto, and a configuration in which a moving average (average value) is calculated in the control section 110 of the detection device 1, and the calculated moving average is transmitted to the first processing device 2 at intervals of the second predetermined time may be adopted.

The first detection system 3 of each of the above-mentioned embodiments is configured such that the detection device 1 includes the transmission section 15, and the first processing device 2 includes the receiving section and is connected through wireless communication. However, the configuration is not limited thereto, and a configuration in which the transmission section and the receiving section are electrically connected through a physical connection such as a connector may be adopted.

The detection device 1 of each of the above-mentioned embodiments is fixed to the skin surface 60 by the adhesive pad 102 but may be configured such that the adhesive pad 102 can be exchanged in consideration of a case where the adhesive strength of the adhesive pad 102 is weakened or the like. In such a case, the detection device 1 is detached from the skin, and then, the adhesive pad 102 is peeled off from the external surface of the case 101, whereby the case 101 and the adhesive pad 102 are separated from each other. Then, a new adhesive pad 102 is attached to the external surface of the case 101, whereby the detection device 1 can be fixed to the skin surface 60 again. According to this configuration, it is possible to continuously use the detection device 1 without throwing it away after using it only once.

The detection device 1 of each of the above-mentioned embodiments is fixed to the skin surface 60 by covering the case 101 with the adhesive pad 102. However, the fixing method including the form is not limited thereto, and for example, the detection device 1 is configured in the form like a watch, and may be fixed to the skin surface 60 of the arm or the like with a band or the like.

The entire disclosure of Japanese Pater t Application No. 2016-074031, filed Apr. 1, 2016 is expressly incorporated by reference herein.

What is claimed is:

1. A detection device, which is a detection device for estimating the occurrence of an allergic reaction, comprising a detection section which noninvasively acquires information of an internal skin or a skin surface as detection data for estimating the allergic reaction.

2. The detection device according to claim 1, wherein the detection section includes
a skin temperature detection section which detects a skin temperature, and
at least one detection section which acquires the detection data other than the skin temperature.

3. The detection device according to claim 2, wherein the at least one detection section which acquires the detection data other than the skin temperature includes any of a blood flow rate detection section which detects a blood flow rate, a vascular diameter detection section which detects a vascular diameter, and a skin thickness detection section which detects a skin thickness.

4. The detection device according to claim 1, wherein the detection section is formed on a flat plate member.

5. The detection device according to claim 1, wherein the device includes a transmission section which transmits the detection data acquired by the detection section to a predetermined device.

6. The detection device according to claim 1, wherein the acquisition of the detection data is performed at intervals within a time period in which an allergic reaction material is present.

7. The detection device according to claim 5, wherein the device includes a time measurement section, and time information measured by the time measurement section is transmitted along with the detection data.

8. The detection device according to claim 5, wherein the device includes a position specification device, and position information and time information by the position specification device are transmitted along with the detection data.

9. A first detection system, comprising
the detection device according to claim 5; and
a first processing device which includes a receiving section that receives the detection data transmitted from the detection device, and a processing section that processes the received detection data and stores the detection data and environmental data corresponding to the detection data in a predetermined memory region.

10. A first detection system, comprising:
the detection device according to claim 6; and.
a first processing device which includes a receiving section that receives the detection data transmitted from the detection device, and a processing section that processes the received detection data and stores the detection data and environmental data corresponding to the detection data in a predetermined memory region.

11. The first detection system according to claim 9, wherein the processing section estimates the presence or absence of an allergic reaction based on the received detection data of the skin temperature.

12. The first detection system according to claim 9, wherein the first processing device includes a position specification device, and acquires the environmental data including position information and time information by the position specification device.

13. The first detection system according to claim 9, wherein the detection data is classified according to predetermined position information and stored.

14. The first detection system according to claim 13, wherein as the predetermined position information, the position information acquired by the position specification device or the position information received along with the detection data is used.

15. The first detection system according to claim 13, wherein the detection data is stored in a chronological order corresponding to the predetermined position information.

16. The first detection system according to claim 9, wherein the detection data is stored when the allergic reaction is estimated to be present.

17. A second detection system, comprising:
a plurality of first detection systems according to claim 9; and
a second processing device which is connected to the plurality of first processing devices constituting the plurality of first detection systems and receives and stores the detection data and the environmental data from the first processing devices.

18. A second detection system, comprising:
a plurality of first detection systems according to claim 11; and
a second processing device which is connected to the plurality of first processing devices constituting the plurality of first detection systems and receives and stores the detection data, and the environmental data from the first processing devices.

19. The second detection system according to claim 16, wherein the second processing device sets a positional region where the allergic reaction is estimated based on the received plurality of pieces of detection data and the environmental data, and transmits the set positional region to the first processing device of each of the first detection systems.

20. The second detection system according to claim 17, wherein the first processing device of the first detection system gives an alarm when acquired current position information is located in a don of the positional region received from the second processing device.
